(19) United States
(12) Patent Application Publication
(10) Pub. No.: US 2021/0307871 A1
(43) Pub. Date: Oct. 7, 2021

(54) NEGATIVE PRESSURE MEDICAL PROCEDURE APPARATUS

(71) Applicant: Adel LLC, Leawood, KS (US)

(72) Inventors: Armand E. Edalati, Kansas City, MO (US); Arya D. Edalati, Kansas City, MO (US); David E. Edalati, Leawood, KS (US)

(73) Assignee: Adel LLC, Leawood, KS (US)

(21) Appl. No.: 17/089,792

(22) Filed: Nov. 5, 2020

Related U.S. Application Data
(60) Provisional application No. 63/005,291, filed on Apr. 4, 2020.
(60) Provisional application No. 63/009,542, filed on Apr. 14, 2020.

(51) Int. Cl. A61B 90/40

(52) U.S. Cl. CPC A61B 90/40; A61B 2217/005

(57) ABSTRACT

A medical containment enclosure defines an internal chamber configured to receive at least part of a patient during a medical procedure, with the enclosure operable to fluidly connect to a suction device. The enclosure includes a containment housing, an access portal, and a suction port. The containment housing at least partly defines the chamber. The access portal defines an orifice that permits chamber ingress and egress. The suction port fluidly communicates with the chamber and is fluidly connectable to the suction device to permit the suction device to draw fluid from the chamber. The access portal restricts fluid flow through the orifice so that fluid removal by the suction device is operable to reduce an internal chamber pressure below an external pressure and thereby restrict aerosols from exiting the chamber via the access portal.

### CROSS-REFERENCE TO RELATED APPLICATIONS

This application claims the benefit of U.S. Provisional Application Ser. No. 63/005,291, filed Apr. 4, 2020, entitled PROTECTIVE SELF CONTAINED INTUBATION APPARATUS, and U.S. Provisional Application Ser. No. 63/009,542, filed Apr. 14, 2020, entitled NEGATIVE PRESSURE PROCEDURE APPARATUS, each of which is hereby incorporated in its entirety by reference herein.

### BACKGROUND

### 1. Field

The present invention relates generally to medical and health care protective equipment. More specifically, embodiments of the present invention concern an enclosure placed over a patient to contain aerosols transmitted from the patient.

### 2. Discussion of Prior Art

Wearable personal protective equipment and other protective devices are well known in the medical arts for protecting doctors, nurses, and other medical staff from exposure to fluids and aerosols transmitted from a patient. Some conventional protective devices, such as curtains, aprons, and shields, are permanently or temporarily installed in an operating space to protect medical staff. For instance, when a patient is laying supine on a gurney or bed, it is known to removably position a splash guard over the patient's head to protect staff from aerosol transmission. Conventional splash guards are particularly used during procedures that require interaction with the patient's airway (e.g., when the patient is being intubated) or other parts of the patient's head or neck.

However, conventional protective devices have various deficiencies. For example, although known splash guards block some transmission paths for aerosols, splash guards still provide other paths for aerosols to escape from the splash guard chamber and into the operating room.

This background discussion is intended to provide information related to the present invention which is not necessarily prior art.

### SUMMARY

The following brief summary is provided to indicate the nature of the subject matter disclosed herein. While certain aspects of the present invention are described below, the summary is not intended to limit the scope of the present invention.

Embodiments of the present invention provide a medical containment enclosure that does not suffer from the problems and limitations of prior art protective equipment, including those devices set forth above.

An aspect of the present invention concerns a medical containment enclosure defining an internal chamber configured to receive at least part of a patient during a medical procedure, with the enclosure operable to fluidly connect to a suction device. The enclosure broadly includes a containment housing, an access portal, and a suction port. The containment housing at least partly defines the chamber. The access portal defines an orifice that permits chamber ingress and egress. The suction port fluidly communicates with the chamber and is fluidly^{,} connectable to the suction device to permit the suction device to draw fluid from the chamber. The access portal restricts fluid flow through the orifice so that fluid removal by the suction device is operable to reduce an internal chamber pressure below an external pressure and thereby restrict aerosols from exiting the chamber via the access portal.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Preferred embodiments of the invention are described in detail below with reference to the attached drawing figures, wherein:

FIG. 1 is an upper side perspective of a medical containment enclosure constructed in accordance with a first preferred embodiment of the present invention, depicting the enclosure placed over the head of a patient to form a chamber for performing a medical procedure on the patient, showing the arms of a healthcare work extended through front access portals of the enclosure;

FIG. 1A is a schematic view of the medical containment enclosure operably associated with a suction system and an oxygen system;

FIG. 2 is an upper side perspective of the medical containment enclosure shown in FIG. 1, with the enclosure including a containment housing, front access portals, side access portals, a suction port, and an oxygen supply port;

FIG. 3 is a lower side perspective of the medical containment enclosure similar to FIG. 2, but taken from the opposite side of the enclosure, with the containment housing including a housing box and a curtain attached to the back wall and side walls of the housing box;

FIG. 4 is an upper rear perspective of the medical containment enclosure shown in FIGS. 1-3;

FIG. 5 is an upper rear perspective of the medical containment enclosure similar to FIG. 4, but showing the curtain drawn outwardly from the housing box to provide access to a chamber of the containment housing;

FIG. 6 is an exploded perspective of the medical containment enclosure shown in FIGS. 1-5, showing the curtain, front access portals, side access portals, suction portion, and supply port exploded from the housing box;

FIG. 7 is an upper front perspective of the curtain shown in FIGS. 1-6;

FIG. 8 is a fragmentary perspective of the medical containment enclosure shown in FIGS. 1-6, showing one of the side access portals attached to the housing box;

FIG. 9 is a fragmentary cross section of the medical containment enclosure taken along line 9-9 in FIG. 8, with the side access portal being cross-sectioned to depict inner and outer flexible layers and an elastomeric layer interleaved between and attached to the flexible layers;

FIG. 10 is a fragmentary exploded perspective of the medical containment enclosure shown in FIGS. 1-6 and 8-9, showing the inner and outer flexible layers and the elastomeric layer of the side access portal exploded from the housing box;

FIG. 11 is a fragmentary exploded perspective of the medical containment enclosure shown in FIGS. 1-6 and 8-10, showing the inner and outer flexible layers and the elastomeric layer of a front access portal exploded from the housing box;

FIG. 12 is a fragmentary exploded perspective of the medical containment enclosure shown in FIGS. 1-6 and 8-11, showing the suction port, tubing, and filter exploded from the housing box;

FIG. 13 is a fragmentary front elevation of the medical containment enclosure shown in FIGS. 1-6 and 8-12, showing elongated front wall openings of the housing box;

FIG. 14 is a fragmentary side elevation of the medical containment enclosure shown in FIGS. 1-6 and 8-13, showing circular side wall openings of the housing box;

FIG. 15 is a rear perspective of a curtain constructed in accordance with a second embodiment of the present invention;

FIG. 16 is a front perspective of the curtain similar to FIG. 15, but taken from the opposite side;

FIG. 17 is a fragmentary front perspective of a medical containment enclosure constructed in accordance with a third embodiment of the present invention, with the enclosure including a housing box, a curtain, a pair of front access portals, and opposite side access portals;

FIG. 18 is a fragmentary rear perspective of the medical containment enclosure similar to FIG. 17, but taken from the opposite side;

FIG. 19 is a fragmentary front elevation of the medical containment enclosure shown in FIGS. 17 and 18, showing elongated front wall openings of the housing box;

FIG. 20 is a fragmentary front elevation of the medical containment enclosure similar to FIG. 19, but showing circular front wall openings substituted in place of the elongated front wall openings shown in FIG. 19;

FIG. 21 is a fragmentary front perspective of a medical containment enclosure constructed in accordance with a fourth embodiment of the present invention, with the enclosure including a housing box, a curtain, a pair of front access portals, and opposite side access portals;

FIG. 22 is a fragmentary rear perspective of the medical containment enclosure similar to FIG. 21, but taken from the opposite side;

FIG. 23 is a fragmentary front perspective of a medical containment enclosure constructed in accordance with a fifth embodiment of the present invention, with the enclosure including a housing box, a curtain, a pair of front access portals, and opposite side access portals, and showing a bronchoscope port in a top wall of the housing box, with the enclosure having a lid to close the bronchoscope port when not in use;

FIG. 24 is a fragmentary rear perspective of the medical containment enclosure similar to FIG. 23, but taken from the opposite side;

FIG. 25 is a fragmentary front perspective of a medical containment enclosure constructed in accordance with a sixth embodiment of the present invention, with the enclosure including a housing box, a curtain, a single front access portal, and opposite side access portals; and

FIG. 26 is a fragmentary rear perspective of the medical containment enclosure similar to FIG. 25, but taken from the opposite side.

The drawing figures do not limit the present invention to the specific embodiments disclosed and described herein. While the drawings do not necessarily provide exact dimensions or tolerances for the illustrated components or structures, the drawings, not including any purely schematic drawings, are to scale with respect to the relationships between the components of the structures illustrated therein.

### DETAILED DESCRIPTION OF THE PREFERRED EMBODIMENTS

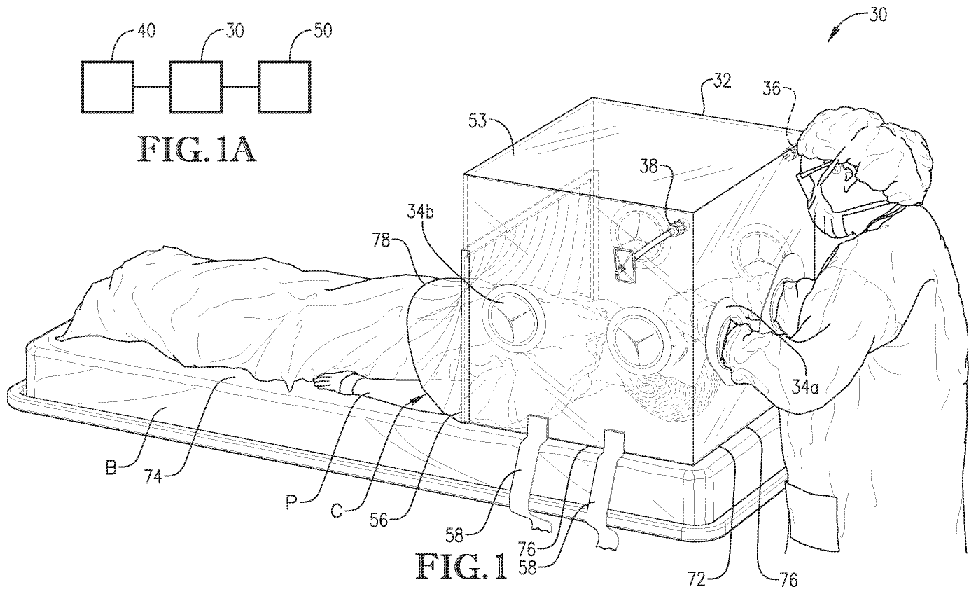

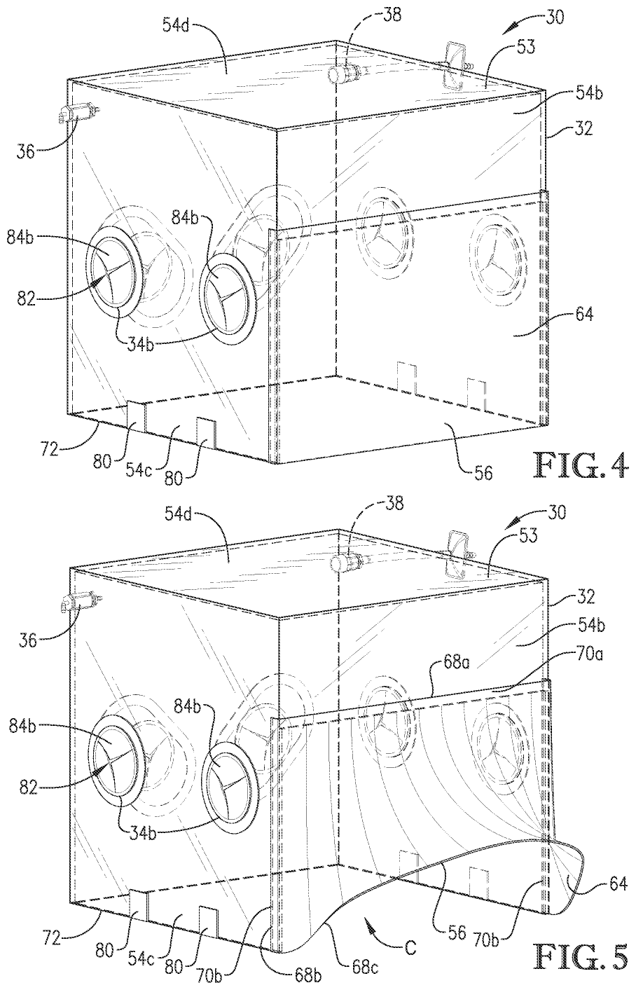

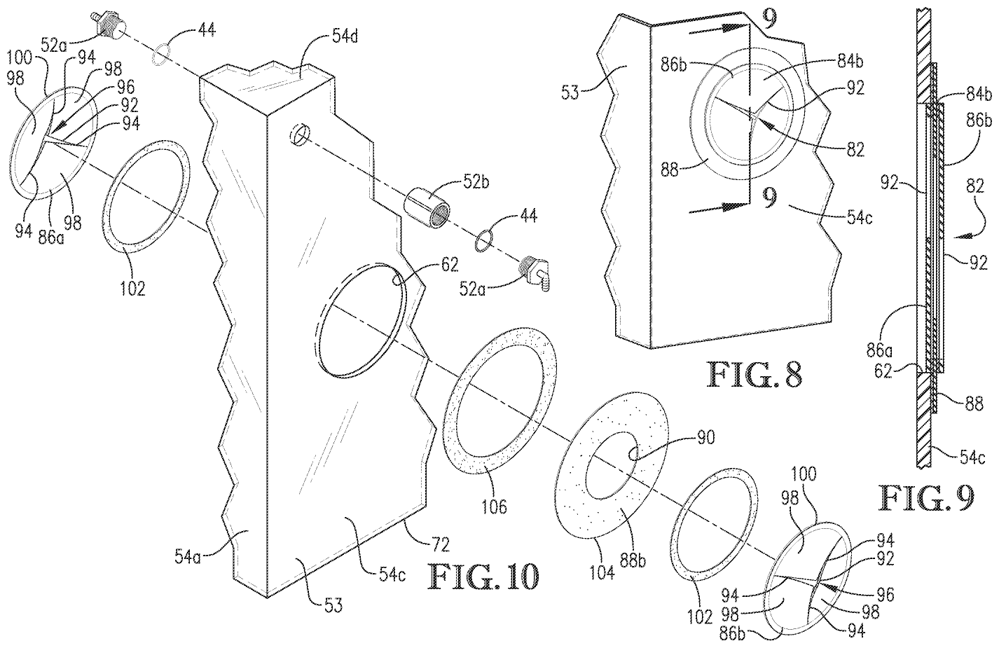

Turning initially to FIGS. 1-14, a medical containment enclosure 30 defines an internal chamber C configured to receive at least part of a patient P during a medical procedure. As will be explained in greater detail, the preferred enclosure 30 is operable to allow one or more health care workers to gain access to the patient P. For instance, the enclosure 30 allows a worker to reach into the enclosure while conducting a procedure or otherwise attending to the patient P. The enclosure may also allow a worker to extend one or more treatment devices, instruments, or other tools (not shown) into the enclosure. Preferably, the enclosure 30 is configured to limit the spread of aerosols from the patient P to health care workers within close proximity to the patient, such as workers within an operating room or other medical treatment room or area.

While the patient P lies supine on a hospital bed B, the enclosure 30 is configured to be placed over the patient's head and upper torso. It is also within the scope of the present invention for the enclosure to be alternatively located to extend over the patient P.

The preferred enclosure 30 broadly includes a containment housing 32, front access portals 34a, side access portals 34b, a supply port 36, and a suction port 38.

### Suction System and Oxygen System

The enclosure 30 is preferably configured to be fluidly connected to a suction system 40, via the suction port 38, so that the suction system 40 can be used to remove contaminants, such as aerosols, from the chamber C (see FIGS. 1 and 1A).

The suction port 38 preferably includes male and female threaded fittings 42a,b. The male fitting 42a extends through the housing 32 and is sealed against the housing 32 with an o-ring 44 (see FIG, 12). The suction port 38 is fluidly connected to the suction system 40 by tubing 46 and an inline filter 48. The filter 48 is preferably a conventional filter for use with a medical vacuum system to remove contaminants, such as aerosols.

As a fluid stream is drawn out of the housing 32 and through the filter 44 by the suction system 40, the filter 44 is operable to remove contaminants, such as aerosols, from the fluid stream. As used herein, an aerosol may include a suspension of fine solid or liquid particles in gas. It will be understood that contaminants may include solids (e.g., fine powder, dust, particulates, etc.), liquids (e.g., liquid drops, droplets, etc.), and/or a gaseous substance. A contaminant volume may be in the form of an aerosol, mist, fog, cloud, vapor, steam, etc.

The suction system 40 preferably includes a conventional powered vacuum system configured for medical purposes. In preferred embodiments, the suction system comprises a wall-mounted unit that generates continuous suction ranging from about fifty (50) mm Hg to about two hundred (200) mm Hg. In the usual manner, the suction system 40 includes, among other things, a vacuum pump (not shown) to draw air and other fluids out of the enclosure 30 via the suction port 38. The suction system 40 also preferably includes a disposal bottle (not shown) for collecting liquid waste. The disposal bottle preferably has a volume ranging from about eight hundred cubic centimeters (800 cc) to about one thousand cubic centimeters (1000 cc).

It will be appreciated that the suction system 40 may be integrally installed as part of a medical room for conducting a patient operation or other treatment. However, an alternative vacuum device may instead be transportable among various treatment rooms. It is within the scope of the present invention for using an alternative vacuum device to draw air and other fluids out of the enclosure 30.

In a similar manner, the enclosure 30 is also configured to be fluidly connected to an oxygen supply system 50, via the supply port 36, so that the system 50 may be used to supply oxygen to the patient P (see FIGS. 1 and 1A). The supply port 36 preferably includes male and female threaded fittings 52a,b. One of the male fittings 52a extends through the housing 32 and is sealed against the housing 32 with an o-ring 44 (see FIG. 12).

The oxygen supply system 50 preferably includes a conventional pressurized supply (not shown) of oxygen, such as one or more pressurized oxygen tanks. The system 50 may also include one or more supply lines (not shown) for carrying oxygen between the pressurized supply and supply port 36 on the enclosure 30. A supply line may extend from the supply port 36 to a respirator (not shown) connected to the patient P.

### Containment Housing

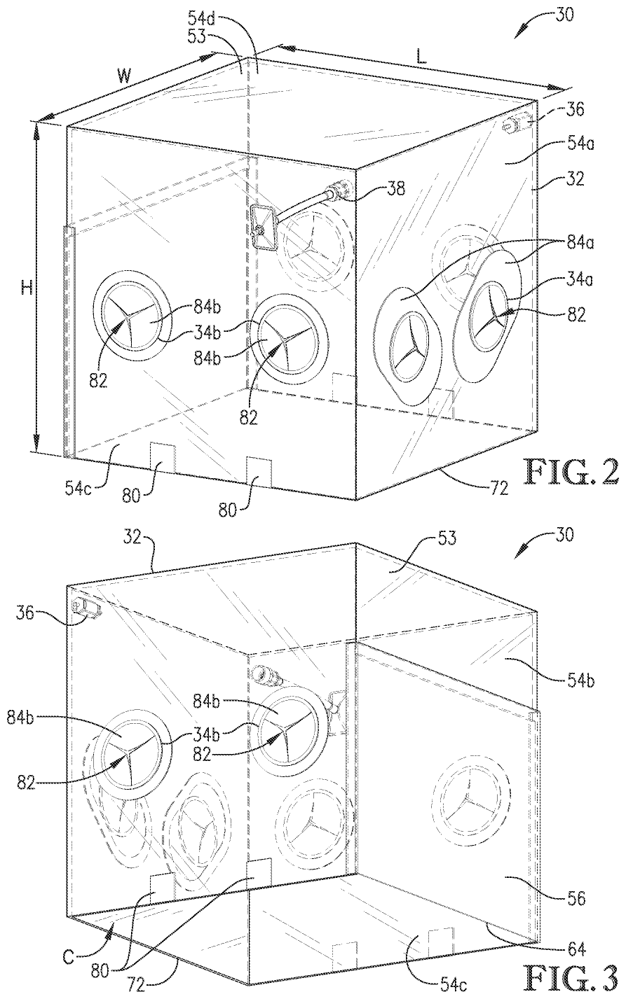

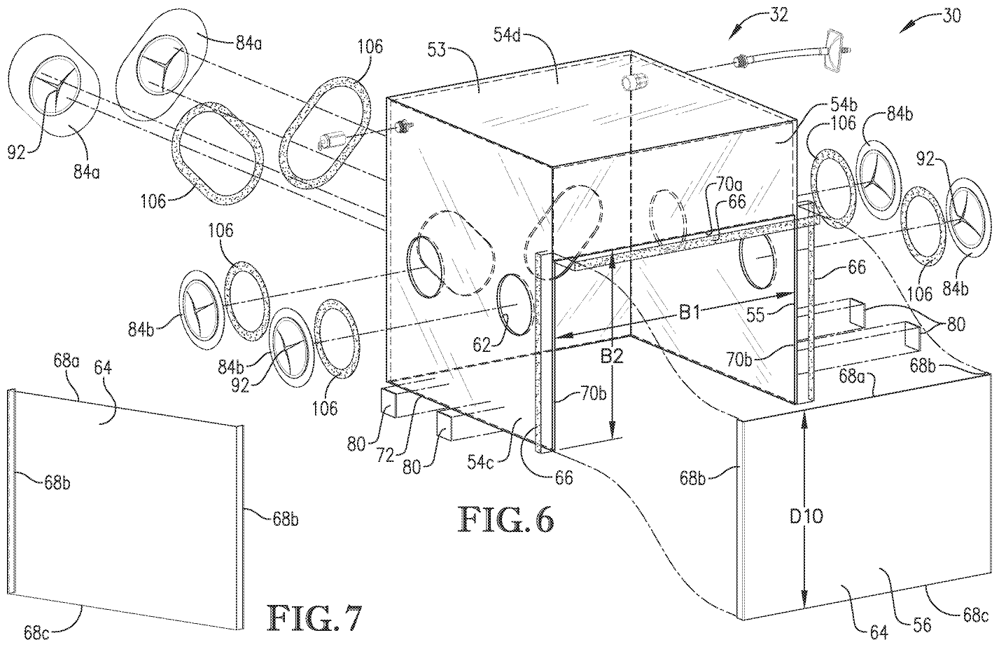

Turning to FIGS. 2-7, the depicted containment housing 32 preferably at least partly defines the internal chamber C, which is operable to receive at least part of the patient P (e.g., during a medical procedure). The containment housing 32 preferably includes a rigid housing box 53 with a front housing wall 54a, back housing wall 54b, side housing walls 54c, and top housing wall 54d (see FIG. 6). The side walls 54c and top wall 54d cooperatively define a box opening 55 that communicates with the chamber C (see FIG. 6).

The containment housing 32 also preferably includes a flexible curtain 56 and fastening straps 58. The curtain 56 is attached to each side wall 54c and the top wall 54d and is configured to span the box opening 55. The walls 54 and curtain 56 cooperatively surround the chamber C and restrict access thereto. As described below, the front and side housing walls 54a, 54c of the containment housing 32 support corresponding access portals 34.

The depicted housing box 53 is constructed with walls 54 in the general shape of a parallelepiped having a width dimension W, height dimension H, and length dimension L (see FIG. 2). The width dimension W, height dimension H, and length dimension L each preferably range from about twelve inches (12″) to about thirty-six inches (36″). More preferably, each of these dimensions is about twenty-four inches (24″).

However, for some aspects of the present invention, one or more of the width, height, and length dimensions of the housing box may fall outside of these ranges. In at least some alternative embodiments, the housing box may be sized to entirely receive the patient. Furthermore, an alternative housing box may be sized to receive the patient and a healthcare worker treating the patient.

Each of the depicted walls 54a,b,c preferably has a generally square shape. However, it is within the ambit of the present invention for the housing box to be alternatively shaped. As will be shown in subsequent embodiments, the housing box may have an alternative width dimension, an alternative height dimension, and/or an alternative length dimension. It will also be understood that one or more walls may present a geometrical shape other than a square shape, such as a rectangle, parallelogram, or trapezoid.

The box opening 53 preferably presents a box opening width B1 and an opening height 132 (see FIG. 6), Preferably, the width B31 ranges from about twelve inches (12″) to about thirty-six inches (36″), and, more preferably, is about twenty-four inches (24″). The height B2 ranges from about six inches (6″) to about twenty-four inches (24″), and, more preferably, is about sixteen inches (16″).

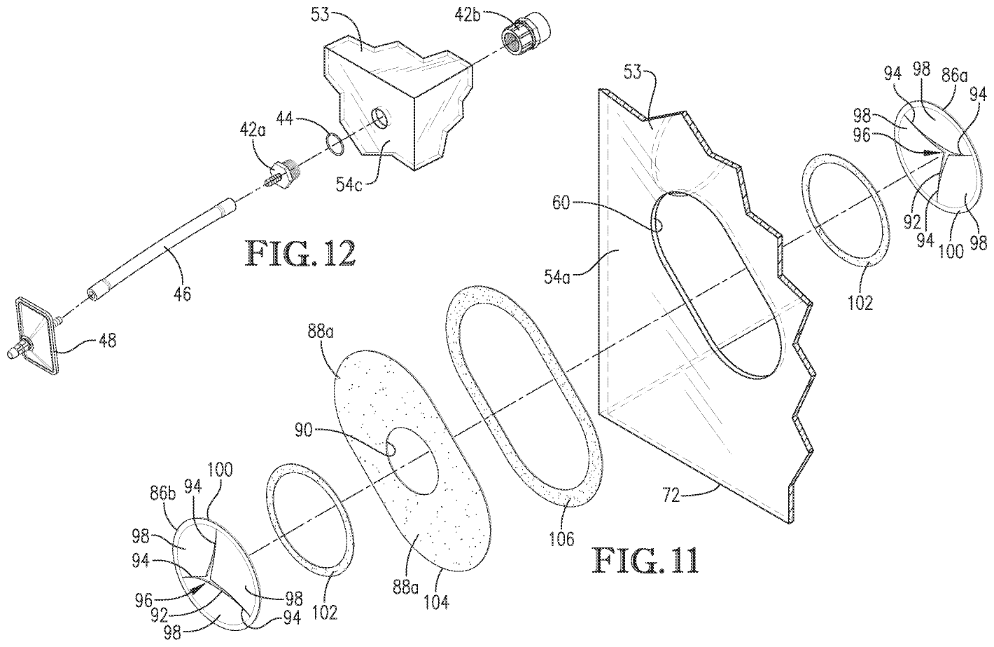

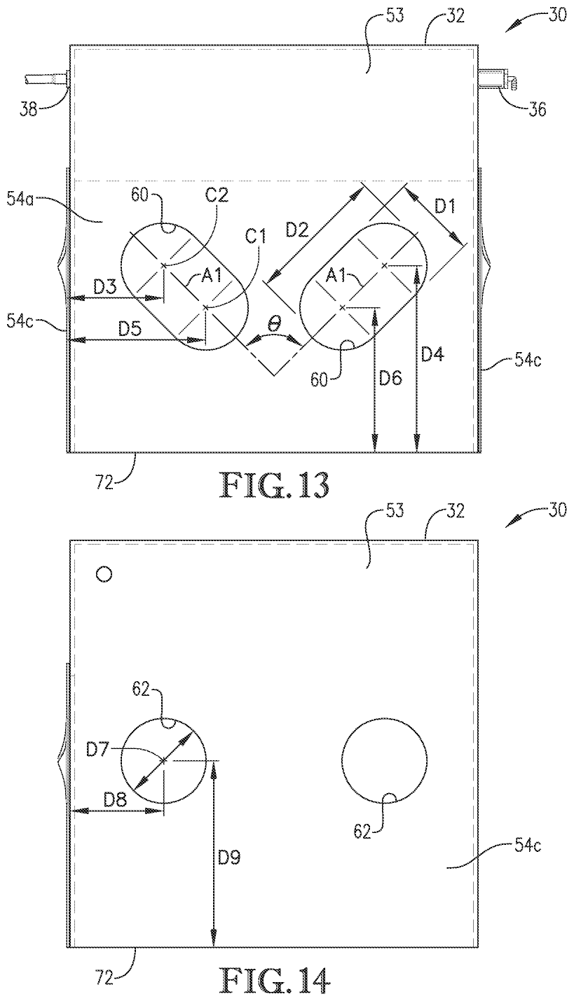

The front wall 54a preferably includes respective elongated front wall openings 60 (see FIGS. 11 and 13). In the depicted embodiment, the front wall openings 60 preferably comprise oval openings that have a width dimension D1 and a length dimension D2 measured along a longitudinal axis A1, with the length dimension D2. being greater than the width dimension D1 (see FIG. 13).

It is within the scope of the present invention for one or more front wall openings to have an alternative elongated shape other than an oval shape (e.g., an elongated slot having a slot axis with a curved, angled, or other nonlinear shape). For some aspects of the present invention, one or more front wall openings may have a circular or polygonal shape.

The width dimension D1 preferably ranges from about four inches (4″) to about eight inches (8″) and, more preferably, is about five inches (5″). The length dimension D2 preferably ranges from about six inches (6″) to about twelve inches (12″) and, more preferably, is about eight inches (8″).

Each front wall opening 60 has an inboard center of curvature C1 that defines an inboard end of the opening 60 and an outboard center of curvature C2 that defines an outboard end of the opening 60 (see FIG. 13). The front wall opening 60 is positioned to define an outer axis side dimension D3, measured relative to the adjacent side of the enclosure, and an outer axis bottom dimension D4 (see FIG. 13). The outer axis side dimension D3 preferably ranges from about four inches (4″) to about ten inches (10″) and, more preferably, is about five and one-half inches (5½″). The outer axis bottom dimension D4 preferably ranges from about six inches (6″) to about eighteen inches (18″) and, more preferably, is about eleven inches (11″).

The front wall opening 60 is also positioned to define an inner axis side dimension D5, measured relative to the adjacent side of the enclosure, and an inner axis bottom dimension D6 (see FIG. 13). The inner axis side dimension D5 preferably ranges from about four inches (4″) to about sixteen inches (16″) and, more preferably, is about eight inches (8″). The outer axis bottom dimension D6 preferably ranges from about four inches (4″) to about sixteen inches (16″) and, more preferably, is about eight and one-half inches (8½″).

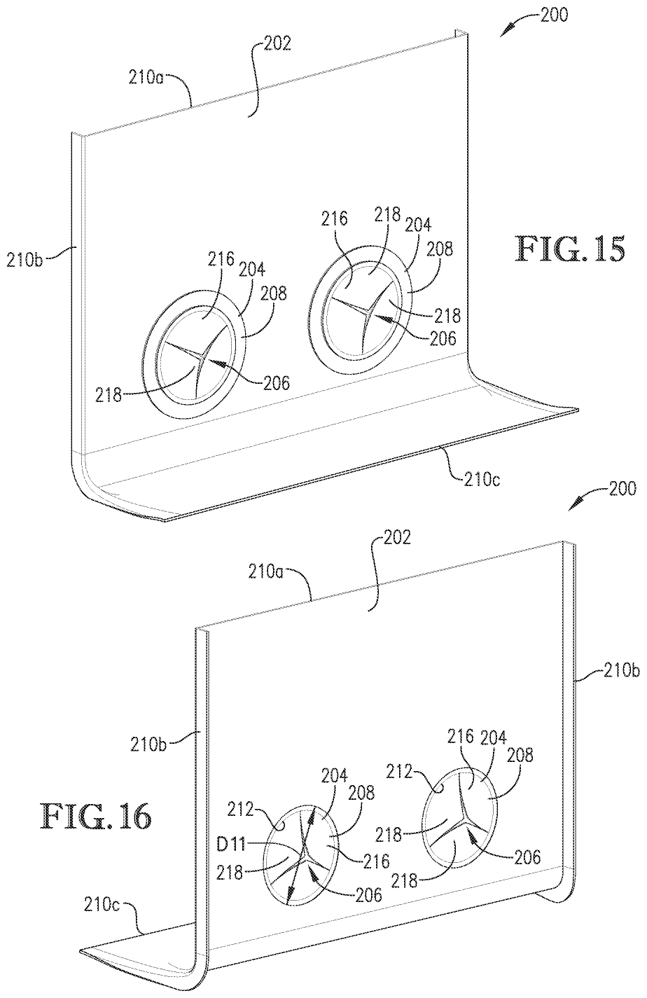

The side walls 54c preferably include respective elongated side wall openings 62 (see FIGS. 15 and 16). Each side wall opening 62 preferably comprises a circular opening that has a diameter dimension D7 (see FIG. 14). It is within the scope of the present invention for one or more side wall openings to have an alternative shape other than a circular shape (such as a polygonal shape). Alternative side wall openings may have an elongated shape, such as an oval shape. Al least one side wall opening may have an elongated slot having a slot axis with a curved, angled, or other nonlinear shape. The diameter dimension D7 preferably ranges from about four inches (4″) to about eight inches (8″) and, more preferably, is about five inches (5″).

Each side wall opening 62 is positioned to define an axis side dimension D8, measured relative to the adjacent side of the enclosure, and an axis bottom dimension D9 (see FIG. 14). The axis side dimension D8 preferably ranges from about two inches (2″) to about twelve inches (12″) and, more preferably, is about five and one-half inches (5½″). The axis bottom dimension D9 preferably ranges from about four inches (4″) to about sixteen inches (16″) and, more preferably, is about eleven inches (11″).

Each front wall opening 60 has a longitudinal axis A1 extending along the corresponding opening length (see FIG. 13). The longitudinal axes A1 of the front wall openings 60 preferably form an included angle è. The angle è preferably ranges from about forty-five degrees (45°) to about one hundred seventy-five degrees (175°). However, for at least some embodiments, the angle è may fall outside of this range.

in preferred embodiments, the walls 54 are generally transparent so that the chamber C (and a portion of the patient therein) can be readily viewed by a person outside the containment housing 32. Similarly, the walls 54 preferably permit a patient P to view people and objects located outside the housing 32. However, one or more walk 54 may include, alternatively or additionally, a translucent material and/or an opaque material.

The walls 54 each preferably comprise a wall structure with a substantially constant wall thickness. The walls 54 preferably include a synthetic resin material that is generally transparent. In preferred embodiments, the material may include a polymer material, such as a polycarbonate, acrylic, and/or polymethyl methacrylate. Although the walls 54 are preferably transparent, it is within the scope of the present invention for at least part of the housing walls to be translucent. For some aspects of the present invention, part of the housing walls may also be opaque.

The walls 54 of the housing 32 are preferably adhered to each other with an adhesive layer (not shown). However, it is within the scope of the present invention for the walls to be rigidly connected relative to each other with various types of interconnecting structures and methods. For instance, at least some of the housing walls may be thermally welded to each other or integrally formed. Furthermore, the housing walls may be attached relative to each other with fasteners (such as bolts, screws, or other threaded fasteners), clamps, straps, bands, etc.

Yet further, it will be understood that at least some of the housing walls may be shiftably connected relative to each other. For example, embodiments of the housing may be provided so that the housing is at least partially collapsible.

Turning to FIGS. 3-7, the curtain 56 is constructed to span the opening 55 of the housing 32 while the enclosure 30 is placed over the patient P. At the same time, the curtain 56 is flexible to permit at least part of the curtain 56, such as a lower margin, to be in conforming contact with the patient P. For instance, the curtain 56 allows the patient P to extend into and out of the chamber C when the containment housing 32 is placed over the patient P.

The depicted curtain 56 includes a curtain wall 64 and elongated fastener strips 66 (see FIG. 6). The curtain wall 64 has a generally rectangular shape and presents an upper attachment margin 68a and side attachment margins 68b (see FIG. 6), The curtain wall 64 also defines a lower margin 68c that is configured to be flexed (see FIG. 6). The curtain wall 64 presents a curtain length dimension D10 (see FIG. 6) measured from the upper margin 68a to the lower margin 68c.

The curtain wall 64 is preferably sized so that the lower margin 68c is generally aligned with the lower margin of the side walls 54c when the curtain wall is attached to the housing box 53. The curtain length dimension D10 is preferably greater than the box opening height B2 so that the upper margin 68a can be attached to the housing box 53 while positioning the lower margin 68c aligned with the lower margin of the side walls 54c. Preferably, the difference between the curtain length D10 and box opening height B2 ranges from about one half inch (½″) to about four inches (4″).

As will be discussed, the lower margin 68c is operable to at least partly conform to and contact the patient P (e.g., by contacting the patient and/or an article donned by the patient). It is also anticipated that the lower margin 68c is capable of at least partly conforming to and contacting the bed B.

The curtain wall 64 presents a curtain width dimension measured from one side margin 68b to the other side margin 68b. The curtain width dimension is preferably greater than the box opening width B1 so that both side margins 68b can be attached to the housing box 53. However, for some aspects of the present invention, the curtain wall may be supported on the housing box without being attached to one of the side margins or without being attached to either side margin. Preferably, the difference between the curtain width dimension and box opening width B1 ranges from about one inch (1″) to about six inches (6″).

The curtain wall 64 preferably comprises a unitary wall structure with a substantially constant wall thickness. The wall thickness of the curtain wall 64 preferably ranges from about one hundredth of an inch ( 1/100″) to about one eighth of an inch (⅛″) and, more preferably, is about one sixteenth of an inch ( 1/16″).

The curtain wall 64 preferably includes a flexible resin material. More preferably, the curtain wall includes an elastomeric material, such as a synthetic rubber material (most preferably, silicone rubber). It will be understood that the curtain wall may include one or more alternative materials (e.g., nitrile rubber or natural rubber) within the scope of the present invention.

In preferred embodiments, at least part of the curtain wall 64 may be transparent so that the chamber C (and a portion of the patient P therein) can be readily viewed through the curtain 56 by a person outside the housing 32. Similarly, the curtain wall 64 may be transparent to permit the patient P to view people and objects located outside the housing 32. However, at least part of the curtain wall may include, alternatively or additionally, a translucent material and/or an opaque material.

The elongated fastener strips 66 of the curtain 56 each preferably include a continuous layer of adhesive material. More specifically, each fastener strip 66 includes a removable double-coated adhesive film tape. Examples of suitable double-coated tape include Model 9425 adhesive tape and Model F9415PC adhesive tape, both supplied by 3M Company.

The fastener strips 66 are arranged end-to-end along, respectively, an upper support margin 70a and side support margins 70b of the walls 54b,c (see FIGS. 5 and 6). The fastener strips 66 also extend along corresponding upper and side margins 68a,68b of the curtain 56.

Preferably, the fastener strips 66 provide continuous adhesive engagement and a hermetic seal between the walls 54b,c and the curtain 56 along the entire axial length of the strips 66. However, the fastener strips may have one or more adjacent strip segments that are separated from one another.

The adhesive material of the fastener strips 66 preferably comprises a temporary adhesive that permits removal of the curtain 56 from the housing walls 54b,c without damaging the curtain 56 or the housing walls 54b,c (e.g., to facilitate sterilization and reuse of the enclosure 30). For some aspects of the present invention, the curtain may be permanently fixed to the housing box, such that the curtain and housing box cannot be separated from each other without damaging at least one of the curtain and the housing box. A permanent connection between the curtain and housing box may be provided by various connection means, such as a permanent adhesive, a weld, or by integrally forming the curtain with at least part of the housing box.

It is within the ambit of the present invention for the housing walls and curtain to be attached to one another by alternative fasteners. For instance, alternative embodiments of the present invention may include other fasteners, such as buttons, hooks, threaded fasteners, zippers, or hook-and-loop material.

The containment housing 32 is preferably positioned so that a lower margin 72 of the walls 54 is supported by the bed B and/or other supporting structure (see FIG. 1). In the disclosed embodiment, substantially the entire lower margin 72 of the walls 54 is depicted as being in contact with the bed B. However, it will be appreciated that the containment housing 32 may be positioned and supported so that only part of the lower margin 72 or no part of the lower margin 72 directly contacts the bed B.

The illustrated bed B is used to support the patient P and has an outer bed surface 74 (see FIG. 1). In various embodiments, it will be understood that the outermost bed surface 74 may be provided by one or more bed elements, such as a fabric sheet, a layered fabric cover, rigid shell, flexible panel, mattress, compressible cushion, and/or pillow. One or more bed elements may contact or otherwise support the walk 54 of the containment housing 32.

Preferably, the lower margin 72 of the walls 54 and the bed surface 74 cooperatively define an interface 76 (see FIG. 1) therebetween. The lower margin 72 substantially conforms to the bed B or other supporting structure on which the containment housing 32 is positioned. The containment housing 32 is preferably configured and/or positioned to minimize any gap, along the interface 76, between the lower margin 72 and the bed B.

For at least some embodiments of the invention, at least part of the lower margin of the walls may sealingly engage the outer bed surface so that the interface is substantially hermetically sealed. However, it will be understood that sealed engagement between the containment housing and bed may not be required to facilitate suitable use of the enclosure to limit the spread of aerosols from the patient P to a health care worker in a treatment room).

Preferably, the lower margin 68c of the curtain 56 forms an interface 78 with the outer bed surface 74, the patient P, and/or another support surface (see FIG. 1). As noted above, the lower margin 68c of the curtain 56 is operable to at least partly conform to and contact the patient P and/or the bed B. By conforming to the patient P and/or the bed B, the curtain wall 64 is operable to minimize any gap, along the interface 78, between the lower margin and an adjacent engaged surface of the patient and/or the bed. In this manner, the curtain 56 facilitates adjustable placement of the enclosure 30 onto the patient P while also reducing aerosol leakage.

Again, the containment housing 32 preferably includes fastening straps 58 to removably secure the containment housing 32 in a position supported on the bed B or another support structure (see FIG. 1). Each strap 58 includes an elongated, flexible strip of material configured to be extended around at least part of the bed B (such as a bed frame or bed mattress) and tensioned when the strap 58 is attached to each of the side walls 54c. Each strap 58 has a strap length that may range from about four feet (4′) to about six feet (6′), although the length may be outside of this range.

The enclosure 30 includes attachment pads 80 that are fixed to corresponding walls 54 for removable attachment to the respective straps 58. The attachment pads 80 are preferably adhered to the walls 54 with an adhesive layer (not shown), although the attachment pads may be attached to the walls by removable fastening elements (e.g., screws, snaps, hooks, etc.) or nonremovable fastening elements.

The straps 58 and attachment pads 80 are removably attachable to one another by removable fastening elements (not shown), such as hook-and-loop material. For instance, the straps 58 may include hook elements, while the attachment pads 80 have complemental loop elements. Alternatively, the straps 58 may include loop elements, while the attachment pads 80 have complemental hook elements.

The scope of the present invention contemplates the use of various types of fasteners to provide either removable or fixed attachment of the straps to the attachment pads and/or the walls. For example, the fastening straps may include discrete fasteners (e.g., buttons, hooks, buckles, snaps, threaded fasteners, and/or zippers). In at least some embodiments, the straps may be connected directly to the housing.

In the depicted embodiment, the suction port 38 is preferably supported by a side wall 54c of the containment housing 32 and fluidly communicates with the chamber C. As noted above, the suction port 38 is fluidly connectable to the suction system 40 to permit the suction system 40 to draw fluid from the chamber C. Similarly, the illustrated supply port 36 is supported by another side wall 54c of the containment housing 32 and fluidly communicates with the chamber C. The supply port 36 is fluidly connectable to the oxygen system 50 so that oxygen can be supplied to the patient P.

The supply and suction ports 36,38 are arranged on opposite side walls 54c of the containment housing 32. In alternative embodiments, it will be appreciated that one or both of the ports may be supported at different locations along the walls 54. For some aspects of the present invention, one or both of the ports may be supported by structure other than the housing walls to facilitate fluid communication with the chamber.

### Access Portals

Turning to FIGS. 2-11, each access portal 34a,b defines an orifice 82 that permits chamber ingress and egress, The access portals 34a,b restrict fluid flow through the corresponding orifice 82 so that fluid removal by the suction system 40 is operable to reduce an internal chamber pressure below an external pressure outside the housing 32, thereby restricting aerosols from exiting the chamber C via the access portal 34a,b.

In the depicted embodiment, each front access portal 34a preferably includes a front wall opening 60 (presented by the front wall 54a) and a shiftable valve-type closure 84a. Each side access portal 34b preferably includes a side wall opening 62 (presented by the respective side wall 54c) and a shiftable valve-type closure 84b. As will be explained, each closure 84a,b is aligned with a respective wall opening 60,62 and attached to the corresponding wall 54 with adhesive.

With respect to the illustrated containment housing 32, the front wall 54a is preferably associated with a pair of front access portals 34a and supports respective closures 84a. Similarly, each side wall 54c is preferably associated with a pair of side access portals 34b and supports respective closures 84b. With this portal configuration, the containment housing 32 permits a healthcare worker to extend both arms through any one of the front or side walls 54a,c. However, as will be shown in subsequent embodiments, alternative containment housings may have an alternative arrangement of access portals.

Each valve-type closure 84a,b preferably includes inner and outer flexible layers 86a,b and a corresponding elastomeric layer 88a,b interleaved between the flexible layers (see FIGS. 10 and 11). As will be described, each closure 84 is shiftable into and out of a closed condition, in which the orifice 82 is at least substantially closed and the closure 84 restricts chamber ingress and egress.

The elastomeric layers 88a,b each comprise a unitary sheet and present a layer opening 90 (see FIGS. 10 and 11) associated with the orifice 82. Each elastomeric layer 88a,b preferably permits expansion and contraction of the opening 90 and the orifice 82. The layer opening 90 presents an opening diameter that preferably ranges from about one half inch (½″) to about two inches (2″).

In the depicted embodiment, each elastomeric layer 88a,b presents a substantially constant wall thickness. The wall thickness of the elastomeric layer 88a,b preferably ranges from about four thousandths of an inch (0.004″) to about five hundredths of an inch (0.050″) and, more preferably, ranges from about eight thousandths of an inch (0.008″) to about sixteen thousandths of an inch (0.016″).

Each elastomeric layer 88a,b preferably includes a flexible elastomeric resin material. More preferably, the layer includes a synthetic rubber material (most preferably, nitrile rubber). It will be understood that the elastomeric layer may include one or more alternative or additional materials (e.g., silicone rubber or natural rubber) within the scope of the present invention.

The illustrated layers 86a,b cooperatively close the orifice 82, while the layer opening 90 of the elastomeric layer 88 remains open. For at least some aspects of the present invention, the elastomeric layer may have an opening that closes when the orifice is closed. The opening 90 preferably has a circular shape when the elastomeric layer 88a,b is in a relaxed condition, although the elastomeric layer opening may have an alternative shape. As used herein, the terms “close” and “closed” may refer to an orifice that is entirely closed, such that the orifice does not permit chamber ingress and egress through the portal, or to an orifice that is substantially closed, such that the orifice restricts chamber ingress and egress and may define a small gap or opening that extends entirely through the portal.

As described previously, each wall opening 60 has an opening length greater than an opening width. The elastomeric layer 88a of each portal 34a preferably permits the portal 34a (and the respective orifice 82) to be at least partly shiftable along the length and/or width of the wall opening 60. To at least some degree, the elastomeric layer 88b of each portal 34b may also permit the portal 34b (and the respective orifice 82) to be at least partly shiftable across the diameter of the wall opening 62.

The inner and outer slotted flexible layers 86a,b are preferably located on opposite sides of the elastomeric layer 88 and partly overlie the elastomeric layer 88. Each layer 86a,b presents respective slots 92 associated with the orifice 82 and permits expansion and contraction of the orifice 82 (see FIGS. 10 and 11).

Each flexible layer 86a,b presents an outer layer diameter that is preferably about five inches (5″). The flexible layer 86a,b also has a unitary wall structure with a substantially constant wall thickness. The wall thickness of each flexible layer 86a,b preferably ranges from about one hundredth of an inch ( 1/100″) to about one eighth of an inch (⅛″) and, more preferably, is about one sixteenth of an inch ( 1/16″).

The flexible layer 86a,b preferably includes a flexible resin material. More preferably, each flexible layer 86a,b includes a synthetic rubber material (most preferably, silicone rubber). It will be understood that the flexible layer may include one or more alternative or additional materials (e.g., nitrile rubber or natural rubber) within the scope of the present invention.

In the illustrated embodiment, each slot 92 is at least partly formed by a pattern of three (3) radial slits 94 that extend radially outwardly relative to a slot center 96 (see FIGS. 10 and 11). In the depicted embodiment, adjacent slits 94 for each flexible layer 86a,b are separated by an angle of about one hundred twenty degrees (120°). Thus, the slot 92 is generally Y-shaped.

The three (3) slots 92 cooperatively define three (3) shiftable flaps 98 of the layer 86a,b. The flaps 98 are shiftable relative to one another and form respective parts of the orifice 82. In the depicted embodiment, the depicted flaps 98 are each preferably shiftable into and out of a closed configuration, corresponding to the closed condition of the closure 84, in which the flaps 98 substantially cover the opening 90.

The principles of the present invention are applicable for one or more flexible layers to have an alternative slot configuration. For instance, the slot may have fewer than three (3) radial slits or greater than three (3) radial slits. Similarly, it will be understood that an alternative layer may have fewer than three (3) flaps or greater than three (3) flaps.

In the illustrated embodiment, the flexible layers 86a,b are preferably arranged so that the slit pattern of one flexible layer 86a,b of the closure 84 is inverted relative to the slit pattern of the other flexible layer 86a,b of the closure 84 (see FIGS. 10 and 11). In other words, each closure 84 has a slit pattern of the inner flexible layer 86a rotated one hundred eighty degrees (180°) relative to the slit pattern of the outer flexible layer 86b. The depicted relative orientation of slots 92 associated with each flexible layer 86a,b of the closure 84 is configured to cooperatively restrict fluid flow through the orifice 82 when the orifice is entirely closed or substantially closed. At the same time, the orientation of slots 92 permits the orifice 82 to be readily opened so that a user can reach through and/or extend an instrument through the orifice 82.

Although each access portal 34 preferably includes inner and outer flexible layers on opposite sides of the elastomeric layer, one or more access portals may have an alternative layered configuration (e.g., to provide suitable closing of the portal while also permitting chamber access). For example, it will be appreciated that the elastomeric layer may not be located between the flexible layers (e.g., where the flexible layers directly overlie each other without another layer therebetween).

An alternative access portal may include more than one elastomeric layer or no elastomeric layer. For an access portal with multiple elastomeric layers, the layers may have layer openings having the same shape or different shapes. For instance, multiple layers may have openings with different opening diameters.

An alternative access portal may include more than two (2) of the slotted flexible layers, a single slotted flexible layer, or no slotted flexible layers. In one such example, the access portal may include the elastomeric layer and a single flexible layer on one side of the elastomeric layer.

The access portals 34a,b are preferably configured to provide removable adhesive attachment of the flexible layers 86a,b and elastomeric layer 88 to one another and to the housing box 53. The access portals 34a,b are preferably removable from the housing box 53 and have layers 86,88 that are separable from one another to permit efficient cleaning of the enclosure 30.

As noted above, the inner and outer flexible layers 86a,b are preferably located on opposite sides of the elastomeric layer 88. Each flexible layer 86a,b is preferably adhered to the elastomeric layer 88 along an outer margin 100 of the layer 86a,b. More particularly, an adhesive layer 102 (see FIGS. 10 and 11) is provided to removably attach the flexible layer 86a,b to the elastomeric layer 88. The adhesive layer 102 preferably provides removable attachment to facilitate sterilization and reuse of the enclosure 30. However, it is within the ambit of the present invention for an alternative attachment or connection to be provided between the layers 86a,b and elastomeric layer 88.

Again, each closure 84 is aligned with a respective wall opening 60,62 and attached to the corresponding wall 54. In particular, the elastomeric layer 88 is preferably adhered to the wall 54 along an outer margin 104 of the layer 88 with an adhesive layer 106 (see FIGS. 10 and 11). Similar to adhesive layer 102, adhesive layer 106 also preferably provides removable attachment to facilitate sterilization and reuse of the enclosure 30. In various embodiments of the invention, the layers 86,88 may be alternatively attached relative to the wall of the containment housing. For instance, the layers 86,88 may be attached to the housing wall, additionally or alternatively, by fasteners, such as buttons, hooks, threaded fasteners, zippers, or hook-and-loop material.

The adhesive layers 102,106 each preferably include a continuous layer of adhesive material. More specifically, each adhesive layer 102,106 includes a removable double-coated adhesive film tape. Examples of removable double-coated tape include Model 9425 adhesive tape and Model F9415PC adhesive tape, both supplied by 3M Company. As used herein, an adhesive is considered “removable” if the adhered components (such as layers 86,88) can be removed from each other without damaging either component.

For some aspects of the present invention, a closure may be configured so that the layers of the closure are permanently fixed to one another, such that the layers cannot be removed from each other without damaging at least one layer. A permanent connection between alternative layers may be provided by various connection means, such as a permanent adhesive, a weld, or by integrally forming the layers with each other.

An alternative closure may be configured so that the layers are permanently fixed to the housing box, such that the layers cannot be removed without damaging at least one layer or the housing box. A permanent connection between the layers and housing box may be provided by various connection means, such as a permanent adhesive, a weld, or by integrally forming the layers with at least part of the housing box.

Each closure 84 is shiftable into and out of a closed condition (see, e.g., FIGS. 2 and 3), in which the orifice 82 is generally closed and the closure 84 restricts chamber ingress and egress. In particular, the flaps 98 of each layer 86a,b associated with the closure 84 cooperate to at least substantially cover the layer opening 90 of the elastomeric layer 88 in the closed position.

Each closure 84 is also shiftable into and out of an open condition, to permit chamber ingress and egress, as shown with the access portals 34a in FIG. 1. For instance, the closure 84 is operable to shift open as a person extends their arm through the orifice 82 of the closure 84. More specifically, as the person extends their arm through the orifice 82, the flaps 98 of each layer 84a,b flex away from each other so that the orifice 82 is enlarged. Similarly, the elastomeric layer 88 flexes so that the layer opening 90 can be enlarged by the person's arm as the arm extends through the orifice 82.

In this manner, the orifice 82 of each access portal 34a,b is preferably adjustably-sized so as to be selectively opened and closed. The closures 84 are preferably constructed so that each orifice 82 is at least partly shiftable along the corresponding wall opening 60,62.

In use, the enclosure 30 is placed over a patient P lying on the bed B prior to treating the patient P. The enclosure 30 is oriented so that the patient's head is located in the chamber C and the curtain 56 lies across the patient's torso. The curtain 56 flexes upwardly and allows the patient P to extend into and out of the chamber C. With the enclosure 30 positioned, the straps 58 may be wrapped around at least part of the bed B to secure the enclosure 30 to the bed B.

The suction port 38 is preferably fluidly connected to the suction system 40 to permit the suction system 40 to draw fluid from the chamber C. With the suction system 40 activated, the suction system 40 is configured to reduce an internal chamber pressure below an external pressure outside the housing 32 and thereby restrict aerosols from exiting the chamber C via the access portal 34a,b.

The supply port 36 is also preferably fluidly connected to the oxygen system 50 so that oxygen can be supplied to the patient P. However, for some aspects of the present invention, the supply port 36 may be closed off (e.g., with a cap or plug element). The difference between ambient pressure and suction pressure is preferably greater than the difference between oxygen supply pressure and ambient pressure. Further, the suction flow rate through the suction port 38 is configured to be greater than the oxygen flow rate through the supply port 36. As a result, the suction port 38 and suction system 40 are configured to ensure a negative chamber gauge pressure, relative to ambient pressure conditions.

During treatment, one or more workers may extend their arm, a treatment device, or other tool through a respective access portal 34. Each access portal 34 is configured to minimize any fluid flow through its orifice 82 while the worker's arm or tool extends through the orifice 82.

### Alternative Embodiments

Turning to FIGS. 15-26, alternative embodiments of the present invention are depicted. For the sake of brevity, the following description will focus primarily on the differences of these alternative embodiments compared to the previous embodiment.

Referring to FIGS. 15 and 16, an alternative curtain 200 is configured for attachment to the side walls and top walls (not shown) of the housing to span the housing opening. Although not depicted, it will be understood that the curtain 200 may used in place of the curtain 56 and attached to the corresponding walls 54b,c of the containment housing 32. The scope of the present invention is also applicable for the curtain 200 to be used as part of alternative containment housing configurations.

The alternative curtain 200 preferably includes a curtain wall 202 and a pair of access portals 204. Each portal 204 defines an orifice 206 to permit chamber ingress and egress. As explained below, the access portal 204 includes a shiftable valve-type closure 208 that is shiftable into and out of a dosed condition, in which the orifice 206 is closed and the closure 208 restricts chamber ingress and egress.

The curtain wall 202 is flexible and presents a generally rectangular shape. The curtain wall 202 presents an upper attachment margin 210a and side attachment margins 210b. The curtain wall 64 also defines a lower margin 210c that is configured to be flexed.

The curtain wall 202 presents a curtain length dimension measured from the upper margin 210a to the lower margin 210c. The curtain length dimension is sized so that the lower margin 210c extends below the lower margin of the housing side walls when the curtain wall is attached to the housing box.

The curtain wall 202 preferably includes a flexible resin material. More preferably, the curtain wall includes an elastomeric material, such as a synthetic rubber material (most preferably, silicone rubber). It will be understood that the curtain wall may include one or more alternative or additional materials (e.g., nitrile rubber or natural rubber) within the scope of the present invention.

The curtain wall 202 preferably presents curtain openings 212 (see FIGS. 15 and 16) of the portals 204. In the depicted embodiment, each curtain opening 212 preferably comprises a circular opening with a diameter dimension D11. The diameter dimension D11 preferably ranges from about four inches (4″) to about eight inches (8″) and, more preferably, is about five inches (5″).

It is within the scope of the present invention for one or more curtain openings to have an alternative shape other than a circular shape (such as a polygonal shape). Alternative curtain openings may also have an elongated shape, such as an oval shape. At least one curtain opening may include an elongated slot having a slot axis with a curved, angled, or other nonlinear shape.

The closure 208 preferably includes an elastomeric layer, which is provided as part of the curtain wall 202. The elastomeric layer of the closure 208 presents the openings 212. The elastomeric layer of the closure 208 permits expansion and contraction of each orifice 206.

The closure 208 also includes a single flexible layer 216 that at least partly overlies the elastomeric layer. The flexible layer 216 is slotted and includes shiftable flaps 218 that define a respective part of the orifice 206. The flaps 218 are shiftable into and out of a closed configuration, corresponding to the closed condition of the closure 208, in which the flaps 218 substantially cover the opening 214. The flexible layer 216 is preferably adhered to the elastomeric layer.

Although the closure 208 includes only a single flexible layer 216, alternative embodiments of the closure may have flexible layers attached to both sides of the elastomeric layer to overlie the respective opening 214.

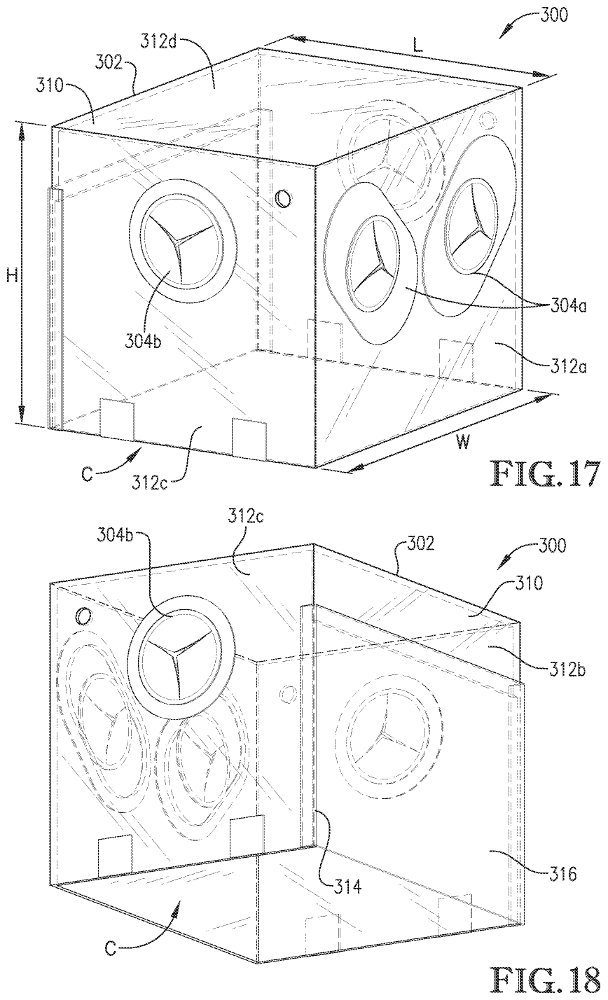

Turning to FIGS. 17-19, an alternative medical containment enclosure 300 defines an internal chamber C configured to receive at least part of a patient P during a medical procedure. The enclosure 300 broadly includes, among other things, a containment housing 302, front access portals 304a, side access portals 304b, a supply port 306, and a suction port 308.

The containment housing 302 preferably includes a rigid housing box 310 with a front wall 312a, back wall 312b, side walls 312c, and top wall 312d. The housing box 310 also presents a box opening 314. The containment housing 302 also preferably includes a flexible curtain 316 that extends across the box opening 314.

The housing box 310 presents a width dimension W, height dimension H, and length dimension L (see FIG. 17). For the illustrated embodiment, the width dimension W is about twenty inches (20″), the height dimension H is about sixteen inches (16″), and the length dimension is about sixteen inches (16″).

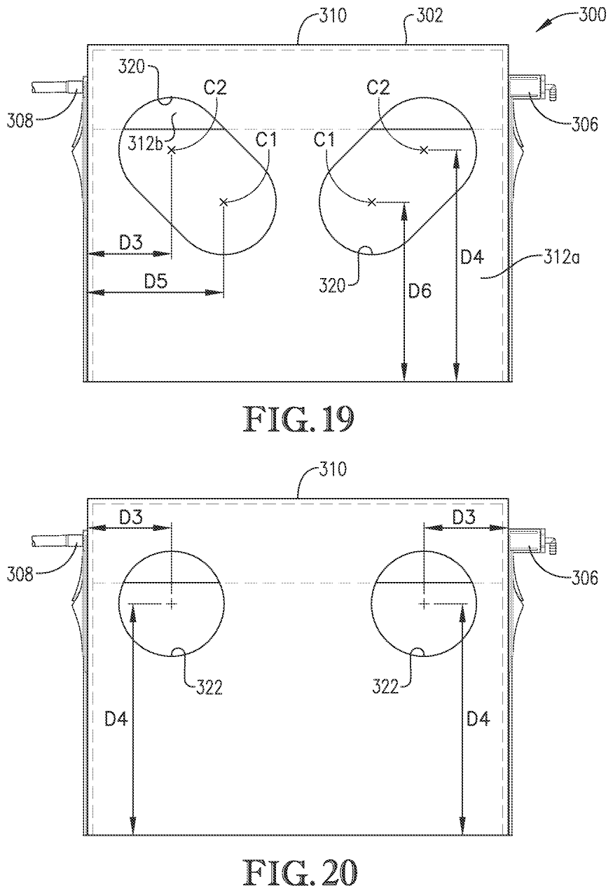

The front wall 312a preferably includes respective elongated front wall openings 320 (see FIG. 19), which have width and length dimensions similar to front wall openings 60. Each front wall opening 320 has an inboard center of curvature C1 that defines an inboard end of the opening 320 and an outboard center of curvature C2 that defines an outboard end of the opening 320 (see FIG. 19). The front wall opening 320 is positioned to define an outer axis side dimension D3 and an outer axis bottom dimension D4 (see FIG. 19).

The outer axis side dimension D3 preferably ranges from about two inches (2″) to about ten inches (10″) and, more preferably, is about four inches (4″). The outer axis bottom dimension D4 preferably ranges from about six inches (6″) to about eighteen inches (18″) and, more preferably, is about eleven inches (11″).

The front wall opening 320 is also positioned to define an inner axis side dimension D5 and an inner axis bottom dimension D6 (see FIG. 19). The inner axis side dimension D5 preferably ranges from about two inches (2″) to about sixteen inches (16″) and, more preferably, is about six and one-half inches (6½″). The outer axis bottom dimension D6 preferably ranges from about four inches (4″) to about sixteen inches (16″) and, more preferably, is about eight and one-half inches (8½″).

In the depicted embodiment, the front wall 312a is preferably associated with a pair of front access portals 304a and supports respective closures. Similarly, each side wall 312c is preferably associated with a single side access portal 304b and supports a respective closure.

Although each front wall opening 320 is preferably elongated and oval shaped, one or both of the front wall openings may be alternatively shaped and/or positioned. For instance, as shown in FIG. 20, the housing box 310 may be modified to replace the oval front wall openings with circular front wall openings 322. The center of each opening 322 is located according to the same side dimension D3 and bottom dimension D4 defined by the wall openings 320.

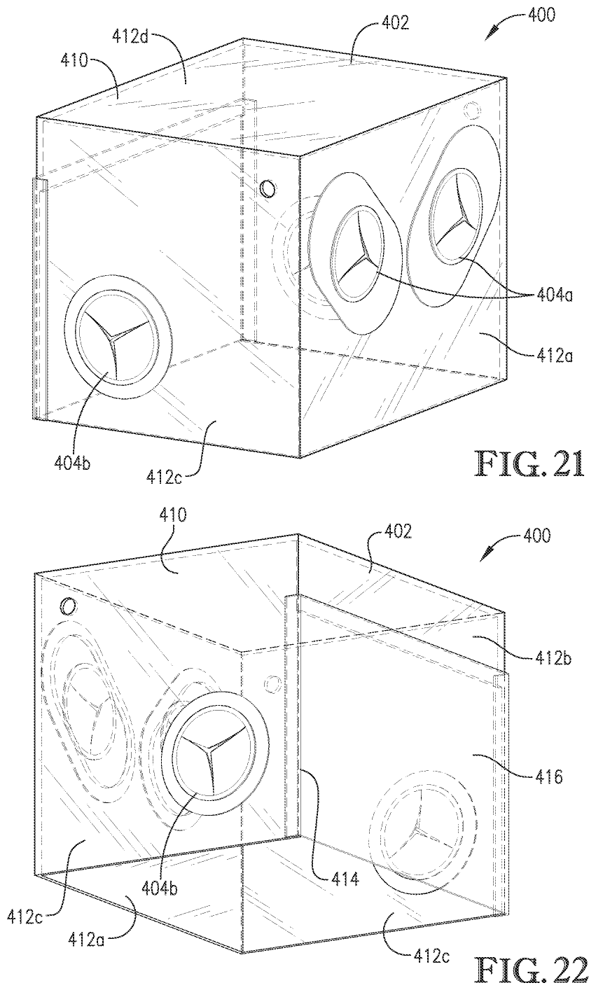

Referring to FIGS. 21 and 22, an alternative containment enclosure 400 includes, among other things, a containment housing 402, front access portals 404a, and side access portals 404b.

The containment housing 402 preferably includes a rigid housing box 410 with a front wall 412a, back wall 412b, side walls 412c, and top wall 412d. The housing box 410 also presents a box opening 414. The containment housing 402 also preferably includes a flexible curtain 416 that extends across the box opening 414. The housing box 410 presents width, height, and length dimensions that are substantially the same as the housing box 310.

In the depicted embodiment, the front wall 412a is preferably associated with a pair of front access portals 404a and supports respective closures. Each side wall 412c is preferably associated with a single side access portal 404b and supports a respective closure. Compared to the enclosure 300, the side access portals 404b of the enclosure 400 are located closer to a lower margin of the curtain 416. It will be appreciated that the location of side access portals 404b may facilitate patient treatment at locations closer to the box opening 414.

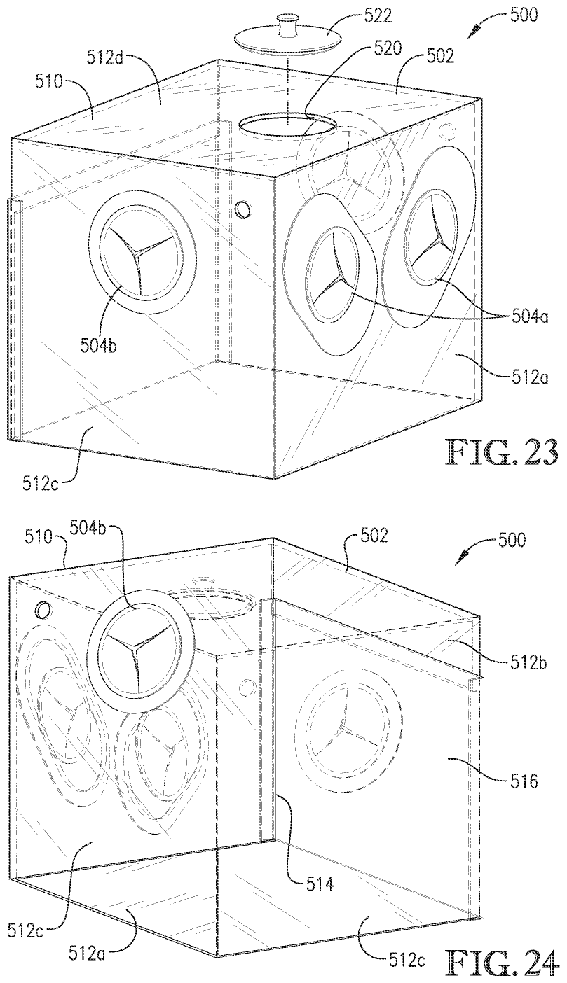

Referring to FIGS. 23 and 24, an alternative enclosure 500 includes, among other things, a containment housing 502, front access portals 504a, and side access portals 504b.

The containment housing 502 preferably includes a rigid housing box 510 with a front wall 512a, back wall 512b, side walls 512c, and top wall 512d. The housing box 510 also presents a box opening 514. The containment housing 502 also preferably includes a flexible curtain 516 that extends across the box opening 514. The housing box 510 presents width, height, and length dimensions that are substantially the same as the housing box 310.

In the depicted embodiment, the front wall 512a is preferably associated with a pair of front access portals 504a and supports respective closures. Each side wall 512c is preferably associated with a single side access portal 504b and supports a respective closure. The top wall 512d also preferably presents a top opening 520 configured to removably receive a lid 522. In preferred embodiments, the top opening 520 is configured to provide a bronchoscope port to operably receive a bronchoscope device (not shown), which may be used to investigate a patient's airway. However, the port may be used for other purposes within the ambit of the present invention

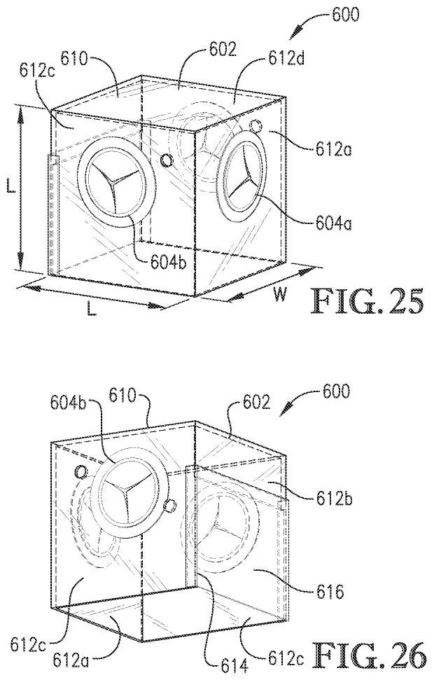

Referring to FIGS. 25 and 26, an alternative enclosure 600 includes, among other things, a containment housing 602, a front access portal 604a, and side access portals 604b. The portals 6040 generally have the same construction and are similar to side portals 34b.

The containment housing 602 preferably includes a rigid housing box 610 with a front wall 612a, back wall 612b, side walls 612c, and top wall 612d. The housing box 610 also presents a box opening 614. The containment housing 602 also preferably includes a flexible curtain 616 that extends across the box opening 614.

The housing box 610 presents a width dimension W, height dimension H, and length dimension L (see FIG. 25). For the illustrated embodiment, the width dimension W is about twelve inches (12″), the height dimension H is about twelve inches (12″), and the length dimension L is about twelve inches (12″).

In the depicted embodiment, the front wall 612a preferably supports a single front access portal 604a, and each side wall 612c preferably supports a single side access portal 604b.

Although the above description presents features of preferred embodiments of the present invention, other preferred embodiments may also be created in keeping with the principles of the invention. Such other preferred embodiments may, for instance, be provided with features drawn from one or more of the embodiments described above. Yet further, such other preferred embodiments may include features from multiple embodiments described above, particularly where such features are compatible for use together despite having been presented independently as part of separate embodiments in the above description.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventors hereby state their intent to rely on the Doctrine of Equivalents to determine and assess the reasonably fair scope of the present invention as pertains to any apparatus not materially departing from but outside the literal scope of the invention as set forth in the following claims.

1. A medical containment enclosure defining an internal chamber configured to receive at least part of a patient during a medical procedure, with the enclosure operable to fluidly connect to a suction device, said enclosure comprising:
a containment housing at least partly defining the chamber;
an access portal defining an orifice that permits chamber ingress and egress; and
a suction port fluidly communicating with the chamber and fluidly connectable to the suction device to permit the suction device to draw fluid from the chamber,
said access portal restricting fluid flow through the orifice so that fluid removal by the suction device is operable to reduce an internal chamber pressure below an external pressure and thereby restrict aerosols from exiting the chamber via the access portal.

2. The medical containment enclosure as claimed in claim 1,
said orifice of the access portal being adjustably-sized so as to be selectively openable.

3. The medical containment enclosure as claimed in claim 2,
said access portal including a shiftable valve-type closure that is shiftable into and out of a closed condition, in which the orifice is closed and the closure restricts chamber ingress and egress.

4. The medical containment enclosure as claimed in claim 3,
said closure including an elastomeric layer that presents an opening associated with the orifice, with the elastomeric layer permitting expansion and contraction of the orifice.

5. The medical containment enclosure as claimed in claim 4,
said closure including a flexible slotted layer that at least partly overlies the elastomeric layer,
said slotted layer including shiftable flaps that define a respective part of the orifice,
said flaps being shiftable into and out of a closed configuration, corresponding to the closed condition of the closure, in which the flaps substantially cover the opening.

6. The medical containment enclosure as claimed in claim 5,
said flexible slotted layer being adhered to the elastomeric layer.

7. The medical containment enclosure as claimed in claim 4,
said closure including inner and outer flexible layers on opposite sides of the ela.stomeric layer,
said inner and outer flexible layers presenting respective slots associated with the and permitting expansion and contraction of the orifice.

8. The medical containment enclosure as claimed in claim 7,
each of said flexible layers including shiftable flaps that define a respective part of the orifice,
said flaps of each flexible layer being shiftable into and out of a closed configuration, corresponding to the closed condition of the closure, in which the flaps substantially cover the opening.

9. The medical containment enclosure as claimed in claim 8,
each of said slots being at least partly formed by a pattern of slits that extend radially outwardly relative to a slot center.

10. The medical containment enclosure as claimed in claim 9,
said flexible layers being arranged so that the slit pattern of one of the flexible layers of the closure is inverted relative to the slit pattern of the other one of the flexible layers of the closure.

11. The medical containment enclosure as claimed in claim 7,
each of said flexible layers being adhered to the elastomeric layer.

12. The medical containment enclosure as claimed in claim 4,
said containment housing including a housing wall along which the access portal is located,
said access portal having a wall opening in the housing wall, with the wall opening having an opening length greater than an opening width,
said elastomeric layer operable to permit the orifice to be at least partly shiftable along the opening length.

13. The medical containment enclosure as claimed in claim 3,
said closure including inner and outer flexible layers that present respective slots associated with the orifice, with the flexible layers permitting expansion and contraction of the orifice.

14. The medical containment enclosure as claimed in claim 13,
each of said flexible layers including shiftable flaps that define a respective part of the orifice,
said flaps of each flexible layer being shiftable into and out of a closed configuration, corresponding to the closed condition of the closure.

15. The medical containment enclosure as claimed in claim 14,
each of said slots being at least partly formed by a pattern of slits that extend radially outwardly relative to a slot center.

16. The medical containment enclosure as claimed in claim 15,
said flexible layers being arranged so that the slit pattern of one of the flexible layers of the closure is inverted relative to the slit pattern of the other one of the flexible layers of the closure.

17. The medical containment enclosure as claimed in claim 1, further comprising:
another access portal defining another orifice that permits chamber ingress and egress,
said containment housing including a housing wall along which the access portals are located.

18. The medical containment enclosure as claimed in claim 17,
said access portals each including a wall opening in the housing wall, with the wall openings each having an opening length greater than an opening width,
each of said orifices being at least partly shiftable along the corresponding opening length.

19. The medical containment enclosure as claimed in claim 18,
said access portals each including a shiftable valve-type closure that is shiftable into and out of a closed condition, in which the orifice is closed and the closure restricts chamber ingress and egress,
said closure including an elastomeric portion that permits shifting of the orifice along the corresponding opening length.

20. The medical containment enclosure as claimed in claim 18,
each of said wall openings having a longitudinal axis extending along the corresponding opening length, with the longitudinal axes of the wall openings forming an oblique angle therebetween.

21. The medical containment enclosure as claimed in claim 1,
said containment housing including a rigid housing wall and a flexible curtain that cooperatively surround the chamber, with the curtain being flexible relative to the housing wall to allow the patient to extend into and out of the chamber when the containment housing is placed over the patient.

22. The medical containment enclosure as claimed in claim 21,
said curtain including another access portal that defines another orifice to permit chamber ingress and egress.

23. The medical containment enclosure as claimed in claim 22,
said another access portal including a shiftable valve-type closure that is shiftable into and out of a closed condition, in which the orifice is closed and the closure restricts chamber ingress and egress.

24. The medical containment enclosure as claimed in claim 22,
said closure of the another access portal including an elastomeric layer that presents an opening associated with the another orifice, with the elastomeric layer permitting expansion and contraction of the another orifice.

25. The medical containment enclosure as claimed in claim 24,
said closure of the another access portal including a flexible slotted layer that at least partly overlies the elastomeric layer,
said slotted layer including shiftable flaps that define a respective part of the another orifice,
said flaps being shiftable into and out of a closed configuration, corresponding to the closed condition of the closure, in which the flaps substantially cover the opening.

26. The medical containment enclosure as claimed in claim 25,
said flexible slotted layer being adhered to the elastomeric layer.
